SEQUENCE LISTING (1 sequence)

SEQ ID NO: 1 - DNA, 1395 bp, unidentified
  source 1..1395: mol_type = genomic DNA
tcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggttccttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacc

(19) United States
(12) Patent Application Publication
(10) Pub. No.: US 2025/0154544 A1
(43) Pub. Date: May 15, 2025

(54) BACILLUS MOJAVENSIS H12 AND APPLICATION THEREOF IN FREMENTATION PRODUCTION OF LOW-MOLECULAR-WEIGHT PULLULAN

(71) Applicant: Shaanxi Institute of Microbiology, Xi'an (CN)

(72) Inventors: Wenjiao Xue, Xi'an (CN); Chen Liu, Xi'an (CN); Jingjing Zhang, Xi'an (CN); Chao An, Xi'an (CN); Qiwen Zhang, Xi'an (CN); Hao Ding, Xi'an (CN); Saijian Ma, Xi'an (CN); Yao Liu, Xi'an (CN); Daidi Fan, Xi'an (CN)

(21) Appl. No.: 19/022,218

(22) Filed: Jan. 15, 2025

(51) Int. Cl. C12P 19/10; C12N 1/20; C12N 9/44; C12P 19/16; C12R 1/07

(52) U.S. Cl. CPC C12P 19/10; C12N 1/205; C12N 9/2457; C12P 19/16; C12Y 302/01041; C12R 2001/07

(57) ABSTRACT

A Bacillus mojavensis H12 is disclosed, wherein the Bacillus mojavensis H12 was deposited in China General Microbiological Culture Collection Center on Apr. 23, 2023, with a deposit number of CGMCC No. 27186 and a classification name of Bacillus mojavensis, and a 16S rDNA sequence is shown in SEQ ID No. 1; a commercial high-molecular-weight pullulan is converted into a low-molecular-weight pullulan by utilizing the pullulanase producing property of Bacillus mojavensis H12, which provides a basis for the industrial production of the low-molecular-weight pullulan.

### SEQUENCE LISTING

The sequence listing is submitted as a XML file filed via EFS-Web, with a file name of “Sequence_Listing. XML”, a creation date of Jan. 10, 2025, and a size of 3,312 bytes. The sequence Listing filed via EFS-Web is a part of the specification and is incorporated in its entirety by reference herein.

### TECHNICAL FIELD

The disclosure relates to the technical field of preparation of low-molecular-weight pullulan and screening of related strains, in particular to a strain of Bacillus mojavensis and its application in fermentation production of low-molecular-weight pullulan.

### BACKGROUND ART

Pullulan is a water-soluble extracellular neutral linear polysaccharide secreted by Aureobasidium pullulans, has good water solubility, biocompatibility, film forming property, cohesiveness and the like, and is widely used in various fields such as foods, medicines, cosmetics and the like. As the application research is advanced, the molecular weight distribution of pullulan is considered as an important parameter for determining its characteristics and application range. Currently, the weight average molecular weight of commercial pullulan is about 1.8*10⁵ to 3.62*10⁵ Da. The low-molecular-weight polysaccharides have better diffusivity in biological tissues and are of significant advantage for use as cosmetic additives and low-calorie food additives.

ZL202010971945.3 discloses a method for producing ultra-low-molecular-weight pullulan by fermentation of Aureobasidium pullulans; ZL202110056362.2 discloses a culture system for preparing low-molecular-weight pullulan and its production process. ZL201710426406.X discloses a mutagenesis strain for efficiently producing low-molecular pullulan and its application. The above patents all use Aureobasidium pullulans as production strains, and use glucose or sucrose as a substrate for fermentation production of low-molecular-weight pullulan, and there have been no reports of preparing the low-molecular-weight pullulan by a microbial converting method.

Therefore, it is an urgent problem for those skilled in the art to provide a strain and a method for converting high-molecular-weight pullulan into low-molecular-weight pullulan.

### SUMMARY

In view of the above, Bacillus mojavensis H12 is screened in the present disclosure, and the commercial high-molecular-weight pullulan is converted into a low-molecular-weight pullulan by utilizing the pullulanase producing property of Bacillus mojavensis H12.

In order to achieve the above purpose, the present disclosure adopts the following technical scheme:

A strain of Bacillus mojavensis H12 is provided, which was deposited in China General Microbiological Culture Collection Center on Apr. 23, 2023, with a deposit number of CGMCC No. 27186 and a classification name of Bacillus mojavensis, and the 16S rDNA sequence is shown in SEQ ID No. 1.

As an inventive concept identical to the above technical solution, the present disclosure also claims the application of Bacillus mojavensis H12 in producing a pullulanase by fermentation, wherein the enzyme activity of the pullulanase is 3.44 U/mL.

As an inventive concept identical to the above technical solution, the present disclosure also claims the application of Bacillus mojavensis H12 in converting high-molecular-weight pullulan into low-molecular-weight pullulan.

As an inventive concept identical to the above technical solution, the present disclosure also claims a method for preparing low-molecular-weight pullulan by utilizing a microbial conversion method, the Bacillus mojavensis H12 is used for the conversion, and the specific process includes the following steps:
(1) Seed liquid preparation: activating the strain H12 frozen and preserved at −80° C. for two successive generations, inoculating the activated strain H12 into a YPD liquid culture medium, and culturing same on a shaker at 28° C. for 18 h at 180 rpm/min to obtain a seed liquid;
(2) Low-molecular-weight pullulan fermentation conversion: adding commercial pullulan into a YPD culture medium, and inoculating same into the seed liquid obtained in step (1) at an inoculum size of 1%, performing a conversion for 48 hours on a shaker at 28° C. and 180 rpm/min to obtain a fermentation broth, centrifuging the fermentation broth and collecting a supernatant, then desalting, decolorizing, drying and crushing the supernatant to obtain the low-molecular-weight pullulan.

It can be seen from the described technical solution that, compared with the prior art, the Bacillus mojavensis strain is separated and purified from the root tissue of the wild Panax bipinnatifidus Seem. in the Qinling Mountains, has good safety, is used for preparing the low-molecular-weight pullulan, has low production cost and controllable molecular weight, and can meet various application requirements of foods, cosmetics and the like. In addition, the production of pullulanase by Bacillus mojavensis has not been reported yet, and the disclosure can also provide a novel source for screening and modifying pullulanase.

### BRIEF DESCRIPTION OF THE DRAWINGS

In order to more clearly illustrate the embodiments of the present disclosure or the technical solutions in the prior art, the drawings that are required to be used in the embodiments or the description of the prior art will be briefly described below, and it is obvious that the drawings in the following description are only embodiments of the present disclosure, and that other drawings can be obtained according to the provided drawings without inventive effort for a person skilled in the art.

FIG. 1 is a graph showing a colony morphology of Bacillus mojavensis H12;

FIG. 2 is a graph showing the gram stain characteristics of Bacillus mojavensis H12;

FIG. 3 is a phylogenetic tree of Bacillus mojavensis H12;

FIG. 4 is a graph showing hemolytic activity of Bacillus mojavensis H12;

FIG. 5 is a graph showing the molecular weight distribution (by GPC) of pullulan before and after fermentation conversion by Bacillus mojavensis H12;

FIG. 6 is a graph showing the IR characteristics of pullulan before and after fermentation conversion by Bacillus mojavensis H12.

### DETAILED DESCRIPTION OF THE EMBODIMENTS

The following description of the embodiments of the present disclosure will be made clearly and completely with reference to the accompanying drawings, in which it is apparent that the embodiments described are only some embodiments of the present disclosure, but not all embodiments. All other embodiments, which can be made by those skilled in the art based on the embodiments of the disclosure without making any inventive effort, are intended to be within the scope of the disclosure.

### Embodiment 1 Isolation and Identification of Strain H12

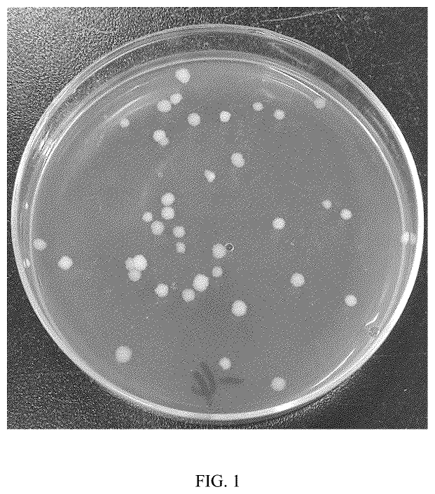

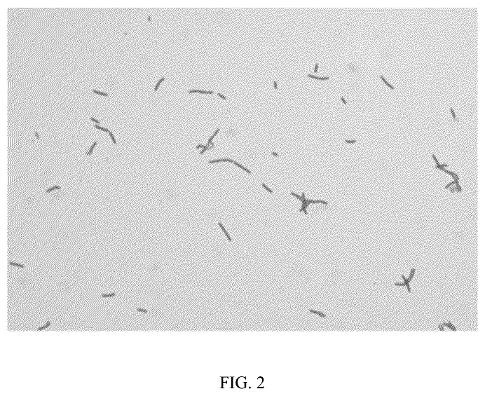

(1) Separation and purification of strains: The root tissue of wild Panax bipinnatifidus Seem. obtained from Qinling Mountains was cut into a size of 1 cm, and was surface-sterilized with 75% ethanol for 1 min, surface-sterilized with 2.5% sodium hypochlorite for 2 min, and surface-sterilized with 75% ethanol for 1 min, respectively, and finally washed with sterile water for 3-5 times to obtain the tissue block, then the tissue block was placed into a pre-sterilized and standby culture dish, then the tissue block was added into a YPD solid culture medium using a sterile forceps. After culturing at 28° C. for 48 h, a single colony was selected for fermentation and coating as shown in FIG. 1, then the gram staining was performed, and the bacterial morphology was observed as shown in FIG. 2. The strain was inoculated into a liquid YPD medium with high-molecular-weight pullulan as a sole carbon source, incubated for 48 h at 28° C., 180 rpm, and the fermentation broth viscosity and OD₆₀₀ were observed. The strain with reduced fermentation broth viscosity and increased OD₆₀₀ were selected and frozen preserved in 25% glycerol at −80° C. for use.

(2) Identification of strains: DNA of strain H12 was extracted using TaKaRa MiniBEST Bacte rial Genomic DNA Extraction kit ver 2.0, and PCR amplification was performed using bacterial 16S rDNA universal primers: an upstream primer 27F: AGAGTTTGATCMTGGC TCAG; downstream primer 1492R: GGTTACTTGTTACGACTT. PCR amplification reaction system (50 μL): 5.0 μL of 10×Taq enzyme buffer, 0.4 μMol/L primer, 4 μL of 200 mmol/L dNTP, 40 ng of DNA template, 1 U of Taq enzyme (TaKaRa), 34 μL of ddH₂O.

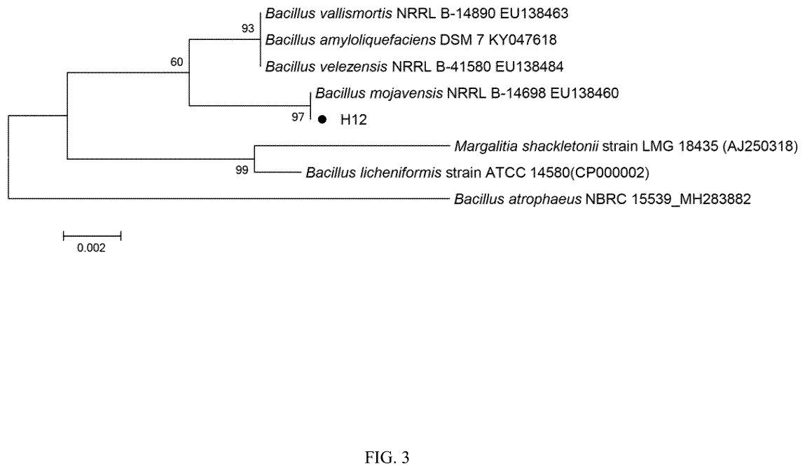

The PCR reaction was amplified as follows: pre-denaturation was performed at 95° C. for 5 min; denaturation was performed at 95° C. for 30 s, annealing was performed at 55° C. for 1 min, extension was performed at 72° C. for 1 min, for 35 cycles; finally, extension was performed for 5 min at 72° C. The reaction was performed on a Bio-Rad PCR apparatus, and the 16S rDNA amplified fragment was detected by 1% agarose gel electrophoresis, and submitted to Shenzhen BGI Gene technology Co., ltd for sequencing. The sequencing result was compared with sequences in GenBank by Blast, the results showed that the 16S rDNA gene sequence (as shown in SEQ ID No. 1) of the strain of the present disclosure had the highest similarity of 97% with the gene sequence of Bacillus mojavensis strain ifo 15718, a phylogenetic tree (FIG. 3) was constructed by MEGA-X software, and it could be known that the strain was Bacillus mojavensis deposited in China General Microbiological Culture Collection Center on Apr. 23, 2023, with a deposit address of No. 3, yard 1, Beichen West Road, Chaoyang District, Beijing, a deposit number of CGMCC No. 27186 and a classification name of Bacillus mojavensis.

### Embodiment 2 Safety of the Strain

After two generations of activation of the strain H12, it was streaked on a blood plate with an inoculating loop, and after upside down culture for 48 hours at 28° C., it was observed whether there was a hemolytic loop. If there was no transparent loop, it would have no β-hemolytic activity, that is, it had no effect on hemolysis or no hemolysis. Commercial strain LGG was used as negative control and Staphylococcus aureus with β-hemolytic activity was used as positive control.

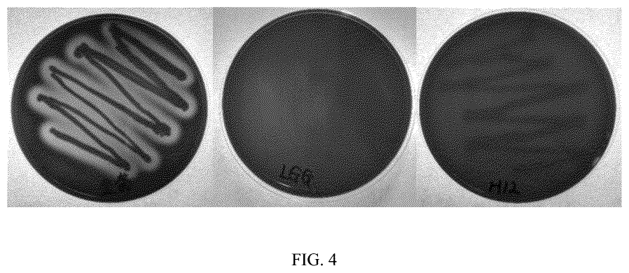

The results show (FIG. 4) that Staphylococcus aureus produced a transparent loop on the blood plate with β-hemolytic activity; H12 and LGG had no transparent loops around them and therefore did not have β-hemolytic activity.

### Embodiment 3 Activity of Pullulanase

The strain H12 frozen and preserved at −80° C. was continuously activated for two generations, inoculated into YPD liquid culture medium with the inoculum size of 1% (v/v), and cultured for 48 hours at the temperature of 28° C. and at the speed of 180 rpm/min to obtain a fermentation broth, then the fermentation broth was centrifuged for 10 min (8000 r/min, room temperature) to obtain a supernatant, the supernatant was taken and the ultrafiltration concentration (10 times volume concentration) was carried out to obtain crude enzyme liquid, and the extracellular pullulanase activity of the strain H12 was measured. 500 μL of the ultrafiltration concentrate and 500 μL of 1 g/L pullulan standard were mixed in a water bath at 55° C. for heat preservation for 30 min. After 2 mL of LDNS was added, the mixture was shaken, boiled in boiling water for 15 min, diluted with 12 mL of water, and shaken well to obtain a reaction solution. The absorbance of the reaction solution was measured at 540 nm in a 0.5 cm cuvette. Standard curve: 0, 0.2, 0.4, 0.6, 0.8 and 1.0 mL of glucose standard solution (1 mg/mL) were respectively taken in a 15 mL test tube, and made up to 1.0 mL with distilled water. 2 mL of DNS reagent was respectively accurately added, heated in a boiling water bath for 2 min, cooled by running water, and made up to a 15 mL with water. Absorbance was measured at 540 nm wavelength. The number of mg/ml of glucose was determined from the standard curve. The sugar content in the sample was determined.

Definition of enzyme activity unit: under the above-specified conditions, the enzyme required for catalytic decomposition of pullulan to produce a reducing sugar equivalent to 1 μmol of glucose per minute was 1 enzyme activity unit (U).

Control: The crude enzyme liquid with the same volume was inactivated in boiling water for 10 min, and the inactivated crude enzyme liquid was added into the reaction system.

The results are shown in table 1 below, indicating that strain H12 produced extracellular pullulanase.

TABLE 1

| | Control group | | Experimental | |
|---|---|---|---|---|
| | U/mL | st | group U/mL | st |
| Concentrated | 1.55 | 0.045 | 3.44 | 0.015 |
| filtrate | | | | |

### Embodiment 4 Conversion of Low-Molecular-Weight Pullulan, Bacillus mojavensis H12 Fermentation Conversion

(1) Seed liquid preparation: the strain H12 frozen and preserved at the temperature of −80° C. was continuously activated for two generations, inoculated into YPD liquid culture medium and cultured for 18 hours on a shaker at the temperature of 28° C. and at the speed of 180 rpm/min, so as to obtain a seed liquid;

The YPD medium included the following components: yeast extract 10 g, protein 20 g, glucose 20 g, pH 5.0-5.5, constant volume to 1 L, and sterilizing at 121° C. for 20 min.

(2) Low-molecular-weight pullulan fermentation conversion: Commercial pullulan was added into a YPD culture medium, and the same was inoculated into the H12 seed liquid obtained in step (1) at an inoculum size of 1%, and the conversion was performed for 48 hours on a shaker at 28° C. and 180 rpm/min to obtain a fermentation broth. The fermentation broth was centrifuged at 8000 rpm/min for 10 min at room temperature and a supernatant was collected. The supernatant was added with twice the volume of 100% ethanol, stirred evenly, and left to stand for 2 h, centrifuged at 8000 rpm/min, 4° C. for 10 min to obtain the pullulan precipitation. Then the pullulan was obtained by drying the pullulan precipitation at 60° C. to constant weight and crushing;

(3) GPC detection: The fermentation broth was sampled at 0, 16, 32, and 48 h of fermentation conversion in step (2), and the pullulan precipitation was obtained as described in step (2). The dried pullulan was prepared into a 1 g/L solution; after filtration with 0.45 μm filter membrane, the solution was chromatographed by gel permeation chromatography to detect the molecular weight distribution characteristics of the substance.

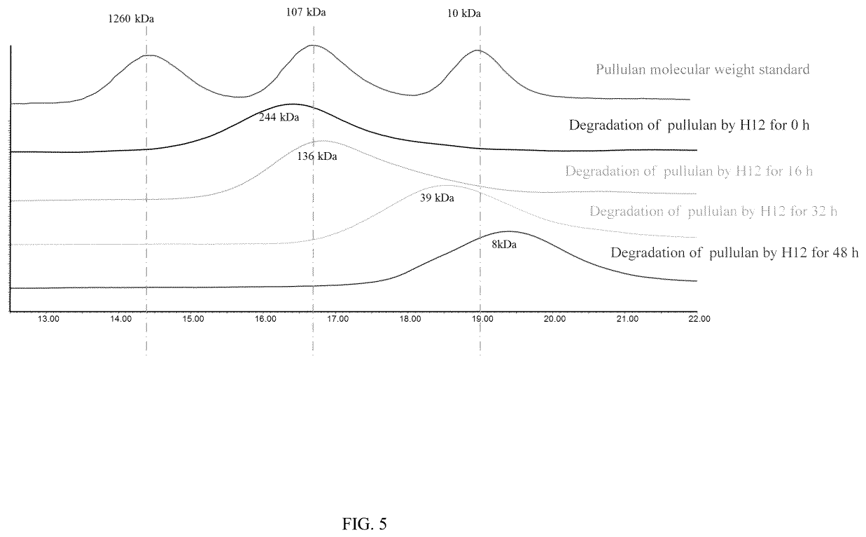

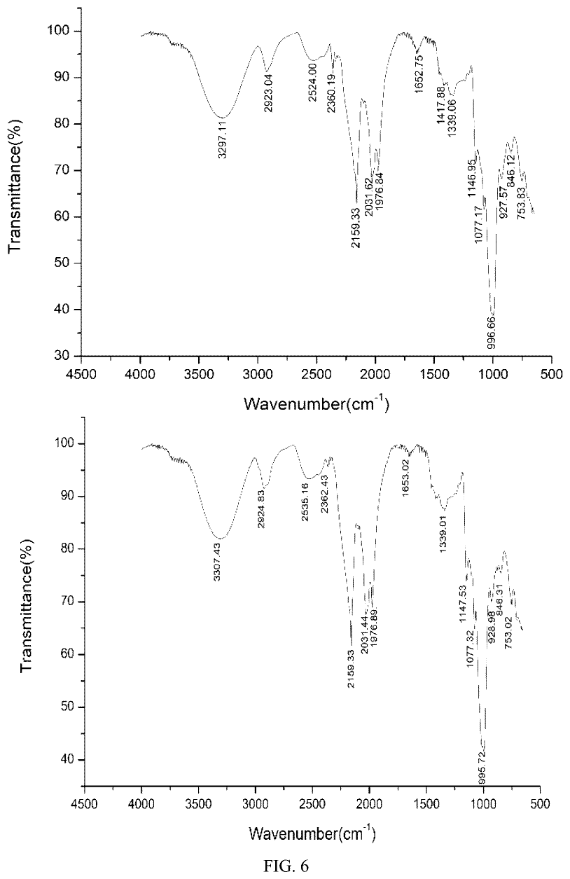

The molecular weight change during conversion of commercial pullulan by strain H12 is shown in FIG. 5. The molecular weight of the commercial pullulan (244 kDa) could reach 8 kDa after 48 hours of conversion, and the recovery rate was 90.2%. The infrared spectra of the pullulan prepared by precipitation before and after conversion were consistent (FIG. 6). The molecular weight and recovery rate of pullulan during the conversion process are shown in table 2 below.

TABLE 2

| | 0 h | 16 h | 32 h | 48 h |
|---|---|---|---|---|
| Molecular Weight | 244 | 136 | 39 | 8 |
| (Mw, kDa) | | | | |
| Pullulan (g/L) | 8.89 | 8.39 | 8.27 | 8.02 |
| Pullulan Recovery | 100% | 94.4% | 93.0% | 90.2% |
| Rate | | | | |

In the present specification, each embodiment is described in a progressive manner, and each embodiment is mainly described in a different point from other embodiments, and identical and similar parts between the embodiments are all enough to refer to each other.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A Bacillus mojavensis H12, wherein the Bacillus mojavensis H12 was deposited in China General Microbiological Culture Collection Center on Apr. 23, 2023, with a deposit number of CGMCC No. 27186 and a classification name of Bacillus mojavensis, and a 16S rDNA sequence is shown in SEQ ID No. 1.

2. An application of the Bacillus mojavensis H12 of claim 1 in producing a pullulanase by fermentation, wherein an enzyme activity of the pullulanase is 3.44 U/mL.

3. An application of the Bacillus mojavensis H12 of claim 1 in converting high-molecular-weight pullulan into low-molecular-weight pullulan.

4. A method for preparing low-molecular-weight pullulan by utilizing a microbial conversion method, wherein the Bacillus mojavensis H12 according to claim 1 is used for conversion, and the specific process comprises:
(1) Seed liquid preparation: activating the strain H12 frozen and preserved at −80° C. for two successive generations, inoculating the activated strain H12 into a YPD liquid culture medium, and culturing same on a shaker at 28° C. for 18 h at 180 rpm/min to obtain a seed liquid;
(2) Low-molecular-weight pullulan fermentation conversion: adding commercial pullulan into a YPD culture medium, and inoculating same into the seed liquid obtained in step (1) at an inoculum size of 1%, performing a conversion for 48 hours on a shaker at 28° C. and 180 rpm/min to obtain a fermentation broth, centrifuging the fermentation broth and collecting a supernatant, then desalting, decolorizing, drying and crushing the supernatant to obtain the low-molecular-weight pullulan.
